(19) United States
(12) Patent Application Publication
(10) Pub. No.: US 2011/0217240 A1
(43) Pub. Date: Sep. 8, 2011

(54) IMAGING NEUROLEPTIC COMPOUNDS

(76) Inventor: Craig Ferris, Holden, MA (US)

(21) Appl. No.: 13/062,108

(22) PCT Filed: Sep. 3, 2009
(86) PCT No.: PCT/US2009/055869

§ 371 Date: May 18, 2011

Related U.S. Application Data
(60) Provisional application No. 61/093,908, filed on Sep. 3, 2008.

(51) Int. Cl. A61K 49/00; A61K 49/06

(52) U.S. Cl. 424/9.2

(57) ABSTRACT

A method for identifying typical and atypical antipsychotics based on their ability to reduce neuronal/glial activity in specific brain regions upon dopaminergic neurotransmission is disclosed.

### FIELD OF THE INVENTION

The present invention is in the field of medicine, more specifically functional neuroimaging, to identify neuroleptic compounds for treating schizophrenia and symptoms associated with schizophrenia.

### BACKGROUND OF THE INVENTION

Schizophrenia is defined as a mental disorder characterized by abnormalities in the perception or expression of reality, suffered by roughly 1% of the world's population irrespective of ethnicity or geography. Schizophrenia is a complex and poorly understood condition, likely caused by a range of factors, including environmental and genetic. There are no known cures for schizophrenia. However, schizophrenia is treatable with antipsychotic medications, which can alleviate the symptoms associated with schizophrenia.

Symptoms of schizophrenia are divided into three broad categories: positive, negative and cognitive symptoms. Positive symptoms are outward manifestations of psychosis and include, for example, thought disorders, delusions and auditory hallucinations. Negative symptoms are the loss or the pronounced reduction of normal traits or abilities, such as flat or blunted affect and emotion, loss or inability to speak, inability to experience pleasure, lack of motivation and social isolation. Cognitive symptoms are problems with attention and the ability to plan and organize.

Antipsychotic medications that are used to treat symptoms of schizophrenia are divided into two classes: typical and atypical antipsychotics. Typical antipsychotic medications, first identified in the 1950s, are quite useful in treating the positive symptoms of schizophrenia, but not the negative or cognitive symptoms. Atypical antipsychotics, on the other hand, are effective in treating all three symptoms of schizophrenia. Unfortunately, both typical and atypical antipsychotics have undesirable side effects. For example, prolonged treatment with typical antipsychotics may lead to tardive dyskinesia, tremors, restlessness, rigidity, and muscle spasms (while failing to treat the negative and cognitive symptoms of schizophrenia). Side effects of atypical antipsychotics include agranulocytosis, weight gain, diabetes and high cholesterol.

Because of the prevalence and the social costs associated with schizophrenia, there is a need to identify new neuroleptic compounds to treat schizophrenia.

### SUMMARY OF THE INVENTION

The disclosure is based, at least in part, on the ability of atypical and typical antipsychotics to increase or decrease brain activity in specific brain regions upon dopaminergic neurotransmission. This discovery has been exploited to develop a method that identifies a typical antipsychotic drug. The method comprises pre-treating a conscious subject with an effective amount of a test neuroleptic; measuring neuronal/glial activity in the hippocampus of the subject by functional imaging; administering a drug that activates dopaminergic neurotransmission to the subject sufficient to alter measurable subject behavior; measuring neuronal/glial activity in the hippocampus of the subject after administration of the drug that activates dopaminergic neurotransmission; and comparing the neuronal/glial activity in the hippocampus of the subject prior to and subsequent to the administration of the drug. A decrease in neuronal/glial activity in the hippocampus indicates that the test neuroleptic is a typical antipsychotic drug.

In certain embodiments, the subject is a mammal, such as a human.

In some embodiments, the drug that activates dopaminergic neurotransmission is a psychostimulant selected from the group consisting of apomorphine, cocaine, amphetamine, methamphetamine, arecoline, methylphenidate, and mixtures thereof.

In another aspect, the disclosure features a method for identifying an antipsychotic drug, comprising pre-treating a conscious subject with an effective amount of a test neuroleptic; measuring neuronal/glial activity in the pituitary gland of the subject by functional imaging; administering a drug that activates dopaminergic neurotransmission to the subject sufficient to alter measurable subject behavior; measuring neuronal/glial activity in the pituitary gland of the subject after administration of the drug that activates dopaminergic neurotransmission; and comparing the neuronal/glial activity in the pituitary gland of the subject prior to and subsequent to the administration of the drug. A decrease in neuronal/glial activity in the pituitary gland indicates that the test neuroleptic is an antipsychotic drug.

In certain embodiments, the subject is a mammal, such as a human.

In some embodiments, the drug that activates dopaminergic neurotransmission is a psychostimulant selected from the group consisting of apomorphine, cocaine, amphetamine, methamphetamine, arecoline, methylphenidate, and mixtures thereof.

In a particular embodiment, the antipsychotic drug is a typical antipsychotic drug or an atypical antipsychotic drug.

In a further aspect of the disclosure, the disclosure features a method for identifying an antipsychotic drug, comprising pre-treating a conscious subject with an effective amount of a test neuroleptic; measuring neuronal/glial activity in the anterior thalamic nuclei of the subject by functional imaging; administering a drug that activates dopaminergic neurotransmission to the subject sufficient to alter measurable subject behavior; measuring neuronal/glial activity in the anterior thalamic nuclei of the subject after administration of the drug that activates dopaminergic neurotransmission; and comparing the neuronal/glial activity in the anterior thalamic nuclei of the subject prior to and subsequent to the administration of the drug. A decrease in neuronal/glial activity in the anterior thalamic nuclei indicates that the test neuroleptic is an antipsychotic drug.

In certain embodiments, the subject is a mammal, such as a human.

In some embodiments, the drug that activates dopaminergic neurotransmission is a psychostimulant selected from the group consisting of apomorphine, cocaine, amphetamine, methamphetamine, arecoline, methylphenidate, and mixtures thereof.

In yet certain embodiments, the antipsychotic drug is a typical antipsychotic drug or an atypical antipsychotic drug.

The following figures are presented for the purpose of illustration only, and are not intended to be limiting.

### BRIEF DESCRIPTION OF THE DRAWINGS

FIGS. 1A-1D are pictoral representations of a neuroanatomical functional magnetic resonance image of an in vivo rat brain (FIG. 1A, untreated) pre-treated with cyclodextrin (FIG. 1B, control vehicle), chlorpromazine (FIG. 1C), and clozapine (FIG. 1D) and then challenged with apomorphine.

FIGS. 2A-2C are bar graph representations of neuronal/glial activity in voxels in the pituitary gland (FIG. 2A), anterior thalamic nuclei (FIG. 2B), and dorsal striatum (FIG. 2C). Voxel numbers between experimental groups were compared using the Newmann-Kuels multiple comparison, non-parametric test statistic. * P<0.05; ** P<0.01.

FIGS. 3A-3C are graphical time course representations showing the percentage change in BOLD signal intensity following ICV administration of apomorphine (arrow). BOLD signal intensity in increased upon apomorphine administration in the pituitary gland (FIG. 3A), the anterior thalamic nuclei (FIG. 3B), and the dorsal striatum (FIG. 3C). Vertical lines at each data point denote the standard error of the mean.

FIGS. 4A-4D are pictoral representations of a neuroanatomical functional magnetic resonance image of an in vivo rat brain (FIG. 4A, untreated) pre-treated with cyclodextrin (control vehicle) (FIG. 4B), haloperidol (FIG. 4C), and olanzapine (FIG. 4D) and then challenged with apomorphine.

FIGS. 5A-5C are graphic representations of neuronal/glial activity in the pituitary gland (FIG. 5A), the anterior thalamic nuclei (FIG. 5B), and the dorsal striatum (FIG. 5C). Voxel numbers between experimental groups were compared using the Newmann-Kuels multiple comparison, non-parametric test statistic. * P<0.05; ** P<0.01.

FIGS. 6A-6D are pictoral representations of a neuroanatomical functional magnetic resonance image of an in vivo rat brain (FIG. 6A, untreated) pre-treated with cyclodextrin (control vehicle) (FIG. 6B), chlorpromazine (FIG. 6C), and clozapine (FIG. 6D) and then challenged with apomorphine.

FIGS. 7A-7D are graphic representations of neuronal/glial activity in the subiculum region of the hippocampus (FIG. 7A), the CA1 region of the hippocampus (FIG. 7B), dentate gyms region of the hippocampus (FIG. 7C), and the CA3 region of the hippocampus (FIG. 7D). Voxel numbers between experimental groups were compared using the Newmann-Kuels multiple comparison, non-parametric test statistic. * P<0.05; ** P<0.01.

FIGS. 8A-8D are pictoral representations of a neuroanatomical functional magnetic resonance image of an in vivo rat brain (FIG. 8A, untreated) pre-treated with cyclodextrin (control vehicle) (FIG. 8B), chlorpromazine (FIG. 8C), and clozapine (FIG. 8D) and then challenged with apomorphine.

FIGS. 9A-9E are graphic representations of neuronal/glial activity in various portions of mesocorticolimbic dopamine pathway: prelimbic (FIG. 9A), accumbens (FIG. 9B), ventral pallidum (FIG. 9C), medial dorsal thalamus (FIG. 9D), and ventral tegmentum (FIG. 9E). Voxel numbers between experimental groups were compared using the Newmann-Kuels multiple comparison, non-parametric test statistic. * P<0.05; ** P<0.01.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the detailed description will be apparent from the following detailed description, and from the claims.

### DEFINITIONS

As used herein, the phrase “test neuroleptic” refers to a compound whose potential antipsychotic effects are unknown within the central nervous system. Test neuroleptics may be typical antipsychotics or atypical antipsychotics.

The phrase “typical antipsychotic” refers to a class of antipsychotic drugs that bind to dopamine D2/D3 receptors, as opposed to other neurotransmitter receptors. Exemplary typical antipsychotics currently used in clinic include, but are not limited to, chlorpromazine, fluphenazine, haloperidol, molindone, thiothixene, thioridazine, trifluoperazine, and loxapine.

As used herein, the phrase “atypical antipsychotic” refers to a class of antipsychotic drugs that bind to both dopamine D2 receptors as well as 5-HT_{1A, 2A} receptors, serotonin receptor subtypes. Exemplary atypical antipsychotics currently used in clinic include, but are not limited to, clozapine, olanzapine, risperidone, quetiapine, ziprasidone, aripiprazole, and paliperidone.

The phrase “measurable subject behavior” as used herein refers to behavioral phenotypes that are observable to an investigator. For example, visible changes in locomotor activity, stereotypy (e.g., paw licking, grooming, etc.), habituation, aggression, emesis, pre-pulse inhibition assays, latent inhibition, social behavior and cognitive skills (e.g., Morris water maze test) may be used to measure alterations in a subject's behavior.

As used herein, the phrase “neuronal/glial activity” is a surrogate marker for measuring cerebral blood flow in a particular brain region. Increased neuronal/glial activity in a particular brain region corresponds to increased blood flow to that brain region to meet the metabolic demands of the neuronal/glial activity. Likewise, a decrease in neuronal/glial activity within a particular brain region correlates to diminished cerebral blood flow due to the decrease in neuronal/glial activity.

As used herein, the phrase “dopaminergic neurotransmission” refers to the release of dopamine into the synapse or agonist binding to dopamine receptors.

The term “psychostimulant” as used herein means any compounds whose abuse is dependent upon mesolimbic and mesocortical dopaminergic pathways. Examples of psychostimulants are, but not limited to, apomorphine, cocaine, amphetamine, methamphetamine, arecoline, and methylphenidate.

As used herein, a “voxel” (volume pixel) is a three-dimensional pixel, or the smallest unit of three-dimensional space in a computer image.

As used herein, a “mammal” may be a human, mouse, rat, guinea pig, dog, cat, horse, cow, pig, or non-human primate, such as a monkey, chimpanzee, baboon or rhesus.

“Schizophrenia” encompasses, but is not limited to, paranoid schizophrenia, disorganized schizophrenia, catatonic schizophrenia and undifferentiated schizophrenia. Schizophrenia may also include bipolar disorder, schizotypal, schizoaffective and drug-induced psychosis.

The methods described herein use neuroimaging techniques to distinguish between typical and atypical antipsychotic drugs based on “fingerprint” brain activities characteristic of atypical and typical antipsychotics during enhanced dopaminergic neurotransmission within particular regions of the brain in conscious subjects, as well as subconscious and unconscious subjects. The methods disclosed can be used to screen chemical compounds for potential activity as neuroleptics, delineate their typical and atypical profiles, and treat schizophrenia and/or psychosis.

The methods disclosed herein may be performed on any subject whose neuronal/glial activity can be measured via standard neuroimaging techniques. In particular, the subjects are mammals, such as primates and humans. One of ordinary skill in the art would be able to identify novel neuroleptics compounds using the methods disclosed herein using primates and humans who suffer from schizophrenia and/or psychosis as subjects.

Alternatively, the methods disclosed may also be performed on animal models of schizophrenia. Neurodevelopmental rat or mouse models of schizophrenia that use neurotoxins to lesion the developing brain (see, e.g., U.S. Pat. No. 5,549,884) may be used as subjects for the methods herein. Similarly, transgenic mouse models of schizophrenia may also be used (see, e.g., Kellendock et al., 2006, Neuron 49:603-15; Hikida et al., 2007, Proc. Natl. Acad. Sci. USA 104:14501-6).

Using the methods described herein, over 100 brain areas of a rat can be screened that are activated by enhanced dopaminergic neurotransmission. A fully segmented rat brain atlas has the potential to delineate and analyze more than 1,200 distinct anatomical volumes within the brain. Because the in-plane spatial resolution of the functional scans (data matrix, 64×64; FOV 3.0 cm) is 486 μm² with a depth of 1,200 μm, many small brain areas (e.g., the nucleus of the lateral olfactory tract) cannot be resolved. Alternatively, if they could be resolved, they would be represented by one or two voxels (e.g., the arcuate nucleus of the hypothalamus). Consequently, small detailed regions are not included in the analysis or are grouped into larger “minor volumes” of similar anatomical classification. For example, in these studies the basal nucleus of the amygdala is listed as a minor volume. This area is a composition of the basomedial anterior part, basomedial posterior part, basolateral anterior part and basolateral posterior part with a composite voxel size of 54.

Due to the non-invasiveness of some neuroimaging techniques, the invention may also be performed on fully conscious subjects, as well as subconscious and unconscious subjects. When imaging awake subjects, it is important to control for motion artifact because any minor head movement will distort the image and will create a change in signal intensity that can be mistaken for stimulus-associated changes in brain activity (Hajnal et al., 1994, Magn. Reson. Med. 31:283-91).

In addition to head movement, motion outside the field of view caused by respiration, swallowing and muscle contractions in the face and neck are other major sources of motion artifact (Yetkin et al., 1996, AJNR Am. J. Neuroradiol. 17(6):1005-9; Birn et al., 1998, Magn. Reson. Med. 40(1):55-60). To minimize motion artifacts when the subject is an animal, studies may be performed using a multi-concentric, dual-coil, small animal restrainer developed specifically for imaging awake rodents (Insight Neuroimaging Systems, LLC, Worcester, Mass.). The reduction in autonomic and somatic measures of arousal and stress improves the signal resolution and quality of the neuroimaging readouts.

### Test Neuroleptic

The present invention provides a method of identifying whether a neuroleptic drug is a typical antipsychotic drug or an atypical antipsychotic drug useful for treating symptoms associated with schizophrenia. The method disclosed herein may be used to screen drug libraries and synthetic peptide combinatorial drug libraries for test neuroleptic drugs. Other drug discovery platforms may also be adapted for use with the present disclosure.

The method recites that an effective amount of a test neuroleptic is administered to the subject. Determining the effective amount for an unknown neurological compound may be readily ascertained by one of ordinary skill in the art. For example, an effective amount may be determined by weight based dosing based on its similarity to other drugs in its class.

A test neuroleptic may cross the blood-brain barrier, i.e., to achieve central nervous system (CNS) permeability, if the test neuroleptic has a molecular mass of less than 500 Daltons (Lipinski et al., 1997, Adv. Drug Del. Rev. 23:3-25). If the test neuroleptic has a mass of 500 Daltons, then a concentration of 500 μg in one liter would be a 1 μM solution. If the subject weighs one kg, one can approximate a total body volume of one liter. Thus, giving this subject 500 μg would achieve a maximum concentration of 1 μM (assuming a homogenous volume of distribution).

### Functional Imaging

Functional imaging as described herein is the study of brain function and activity based on the analysis of data acquired using brain imaging modalities. Such brain imaging modalities are, but are not limited to, functional magnetic resonance imaging (fMRI), positron emission tomography (PET), single photon emission computed tomography (SPECT), optical imaging, thermal imaging, electroencephalogram (EEG), magnetoencephalogram (MEG) and two-photon laser-scanning microscopy. Due to its non-invasive nature, quick scan times, and image resolution, blood oxygen-level dependent (BOLD) fMRI is most commonly used for neuroimaging experiments.

### Functional Magnetic Resonance Imaging

BOLD fMRI measures the blood flow to the local vasculature that accompanies brain activity. Blood oxygen is released to active neurons and glia at a greater rate than to inactive neurons and glia. The difference in magnetic susceptibility between oxyhemoglobin and deoxyhemoglobin, and thus oxygenated or deoxygenated blood, leads to magnetic signal variation which can be detected using an MRI scanner. MRI scanners are available at Oxford Instrument (Oxford, U.K.).

However, there are times where an increase in BOLD signal may be caused without any neuronal/glial activity, e.g., a CO₂ challenge. Upon inhalation of CO₂, the arteries dilate and this in turn causes an increase of blood flow to the area. The increase of blood flow to the area is not caused by an increase of neuronal/glial activity but is a consequence of CO₂ challenge. However, this “false-positive” result can be circumvented by measuring the cerebral blood flow (CBF) of the proton molecules in the water molecules of blood as the tracer. Functional MRI can thus measure direct changes in CBF, irrespective of neuronal/glial activity.

Another fMRI method employs paramagnetic contrast agents that alter local magnetic susceptibility and enhance the sensitivities of fMRI signals. Using this method, regional and global changes in cerebral blood volume (CBV) can be detected. Examples of paramagnetic contrast agents include, but are not limited to, metalloporphyrins such as gadolinium-based contrast agents (including, but not limited to, Omniscan™ or gadodiamide (GE Healthcare, UK), Magnevist™ or gadopentetate dimeglumine (Berlex Laboratories, Inc., Trenton, N.J.), Optimark™ or gadoversetamide (Mallinckrodt Inc., St. Louis, Mo.)) and monocrystalline iron oxide nanocolloid (MION) (The Center for Molecular Imaging Research, Massachusetts General Hospital, Charlestown, Mass.). Other paramagnetic contrast agents include, but are not limited to, gadopentetic acid, gadoteric acid, gadoteridol, mangafodipir, ferric ammonium citrate, gadobenic acid, gadobutrol, gadoxetic acid, Photofrin™ (porfimer sodium), gold-coated and dextran-coated MIONs.

Subject motion is an issue in fMRI data analysis; even the slightest movement during the scan can displace voxel location corresponding to a distinct physical area. Unlike human fMRI, this issue is more prevalent in small animals like rats, because voxel size is much larger than physical (anatomical) area in the brain. The change in signal intensity due to motion can be greater than the BOLD signal, especially at the edge of the brain and tissue boundaries which essentially leads to artifact in the activation map. To avoid this, “motion correction” has become common preprocessing step in fMRI data analysis. Commonly used motion correction tools include automated image registration (AIR) (Woods et al., 1992, J. Comput. Assist. Tomogr. 16:620-33; Woods et al., 1998, J. Comput. Assist. Tomogr. 22:139-52; Woods et al., 1998, J. Comput. Assist. Tomogr. 22:153-65), analysis of functional neuroimages (AFNI) (Cox, 1996, Comput. Biomed. Res. 29(3):162-73), and statistical parametric mapping (SPM) realign tools (Friston et al., 1996, Magn. Reson. Med. 35:346-55).

However, motion correction may induce spurious activation in motion-free fMRI data (Freire and Mangin, 2001, NeuroImage 14:709-22). This artifact stems from the fact that activated areas behave like biasing outliers for the difference of square-based measures usually driving such registration methods. This problem is amplified in case of small mammals where the BOLD signal change can be 10% or greater over baseline. If motion parameters are included in the general linear model for event-related data, it makes little difference if motion correction is actually applied to the data (Johnstone et al., 2000, Hum. Brain Map 27:779-88).

Image resolution using fMRI depends on the strength of the magnet. Magnets employed for fMRI studies range from 1.5 Tesla (T) to 11.7 T. The more powerful the magnet, the greater the resolution of the image. For brain imaging studies, the typical magnet strength is about 4.7 T and 7.0 T (GE Healthcare, U.K.; Bruker BioSpin, U.S.). Using a magnet field strength greater than 7.0 T may be problematic as there are limitations with high magnetic field strengths. For example, stronger magnetic field strengths shorten the T2 relaxation time, thereby making it difficult to delineate boundaries in fMRI studies that favor T2-weighted sequences.

### Positron Emission Tomography

Positron emission tomography (PET) also measures CBF using radiolabeled compounds. This invasive imaging modality takes advantage of the unstable positron-emitting isotopes (for example, ¹⁵O and ¹¹C) incorporated in radiolabeled water or glucose. When injected into the bloodstream, the radiolabeled water or glucose is delivered to the active neurons and glia. As the unstable isotope decays, a positron is emitted and eventually collides with an electron, thereby emitting two gamma rays, which are then measured using gamma ray detectors. By reconstructing the sites of the positron-electron collisions, the location of active regions can be imaged. Cyclotrons, which are used to produce the positron-emitting isotopes, and PET imaging scanners may be purchased from GE Healthcare (U.K.).

### Single Photon Emission Computed Tomography

Single photon emission computed tomography (SPECT) imaging also measures CBF using radiolabels that need to be injected into the subject. Red blood cells pick up and distribute the injected radiolabel (for example, ¹²³I-labeled iodoamphetamine) throughout the body, specifically to areas of high metabolic activity. As the radiolabel decays, photons are emitted and detected to recreate a three-dimensional image of neuronal/glial activity. Compared to fMRI or PET, image resolution from SPECT is low and is thus better suited to image large regions of the brain as opposed to finer features within. Because radiolabeled tracers, rather than positron-emitting isotopes, are used, a cyclotron is not needed. Gamma ray detectors, similar to the ones used in PET imaging, are then used to detect and image the neuronal/glial activity.

### Electroencephalogram

Electroencephalograms (EEGs) measure the electrical activity of the brain as a measure of time varying spontaneous potentials through a number of electrodes attached to the scalp. The information from the electrical activity obtained through EEG analysis is recorded as sets of traces of the amplitude of spontaneous potentials over time. While EEGs can capture oscillations created by brain electric potentials from the 10 millisecond to 100 millisecond range, its spatial resolution is quite poor. When the subjects are animals, surgery is typically required to mount the electrodes directly onto the animal's skull. Pinnacle Technology, Inc. (Lawrence, Kans.) manufactures a rat and mouse EEG system suitable for use with the method disclosed.

### Magnetoencephalogram

Whereas EEG measures electrical activity of the brain, magnetoencephalograms (MEGs) measure the magnetic field changes associated with neuronal firing. Superconducting magnetic detectors detect rapidly changes in magnetic fields and translate them into detectable alterations in electric current. Like EEG, MEG also has superior temporal resolution and poor spatial resolution. Pinnacle Technology, Inc. (Lawrence, Kans.) also sells MEG systems for rodents.

### Dopaminergic Neurotransmission and Subject Behavior

The dopamine hypothesis of schizophrenia suggests that hyperactivation of dopaminergic neurotransmission causes the symptoms of schizophrenia (Seeman, 1987, Synapse 1:133-52). Support for this hypothesis stems from the fact that antipsychotics bind to the dopamine D2 receptor and prevent dopamine neurotransmission. Psychostimulants such as amphetamines enhance dopaminergic neurotransmission by activating the dopamine receptors.

A sufficient amount of a psychostimulant administered to a subject will alter the subject's behavior measurably. These changes in the subject's behavior are objectively measurable and quite well known to one of ordinary skill in the art. Motor activity, social interactions, and cognitive behavior are examples of subject behaviors that can be objectively measured after administration of a psychostimulant. Below, some known behavioral tests for abnormal behavior in rats are described.

The tail-pinch or immobilization test involves applying pressure to the tail of the animal and/or restraining the animal's movements, subsequently measuring, for example, motor activity, social behavior, and cognitive behavior, and statistically analyzing the behaviors measured. (See, e.g., D'Angic et al., 1990, Neurochem. 55:1208-14).

The prepulse inhibition of startle response test involves exposing the animal to a sensory stimulus, objectively measuring the startle responses of the animal to similar acoustic or tactile stimuli, and statistically analyzing the behaviors measured. (See, e.g., Geyer et al., 1990, Brain Res. Bull. 25:485-98).

The social interaction test involves exposing the rat to other animals in a variety of settings, objectively measuring subsequent social behaviors such as, for example, touching, climbing, sniffing and mating, and statistically analyzing the behaviors measured. (See, e.g., File et al., 1985, Pharmacol. Bioch. Behav. 22:941-4; Holson, 1986, Phys. Behav. 37:239-47).

The learned helplessness test involves exposure to stresses, e.g., noxious stimuli, which cannot be affected by the behavior of the animal and subsequently exposing the animal to a number of behavioral paradigms. The behavior of the animal is statistically analyzed using standard statistical tests. (See, e.g., Leshner et al., 1979, Behav. Neural Biol. 26:497-501).

The Morris water-maze test comprises learning spatial orientations in water and subsequently measuring the animal's behaviors, such as, for example, by counting the number of incorrect choices. The behaviors measured are statistically analyzed using standard statistical tests. (See, e.g., Spruijt et al., 1990, Brain Res. 527:192-7).

The passive avoidance or shuttle box test generally involves exposure to two or more environments, one of which is noxious, and a choice must be learned. Behavioral measures include, for example, response latency, number of correct responses, and consistency of response. (See, e.g., Ader et al., 1972, Psychon. Sci. 26:125-8; Holson, 1986, Phys. Behav. 37:221-30).

The invention is further illustrated by the following examples. The examples are provided for illustrative purposes only. They are not to be construed as limiting the scope or content of the invention in any way.

### EXAMPLES

### Identification of Brain Areas Responsive to Both Typical and Atypical Antipsychotics Using fMRI

A. Methods

Live Animal Imaging

To image the brain activity of live rats (Charles River Laboratories, Wilmington, Mass.), the rats were anesthetized with 2-3% isoflurane (Abbott Laboratories, North Chicago, Ill.). Nine (9) rats were used for each experimental condition. A topical anesthetic of 10% lidocaine gel was applied to the skin and soft tissue around the ear canals and over the bridge of the nose. A plastic semi-circular headpiece with blunted ear supports that fit into the ear canals was positioned over the ears. The head was placed into a cylindrical head holder with the rat's canines secured over a bite bar and ears positioned inside the head holder with adjustable screws fitted into lateral sleeves. An adjustable, receive-only surface coil built into the head holder was pressed firmly on the head and locked into place. The body of the rat was placed into a body restrainer. The body restrainer “floats” down the center of the chassis connecting at the front and rear end-plates and buffered by rubber gaskets. The head piece locks into a mounting post on the front of the chassis. This design isolates all of the body movements from the head restrainer and minimizes motion artifact. Once the rat was positioned in the body holder, a transmit-only volume coil was slid over the head restrainer and locked into position.

Acclimation to Imaging Protocol

To address the issue of imaging restrained, fully conscious animals, protocols have been developed for acclimating animals to the environment of magnetic resonance scanners and imaging procedures leading to a reduction in stress hormones levels and measures of autonomic activity regulated by the sympathetic nervous system (Stoffman et al., 2005, Neurosurgery 57(2):307-13; Zhang et al., 2000, Brain Res 852(2):290-6). Acclimation protocols have been used to prepare awake animals for a range of behavioral, neurological and pharmacological imaging studies, including sexual arousal in monkeys (Ferris et al., 2004, J Magn Reson Imaging 19(2):168-75), generalized seizures in rats and monkeys (Tenney et al., 2004, Epilepsia 45:1240-7; Tenney et al., 2003, Epilepsia 44:1133-40), and exposure to psychostimulants like cocaine (Febo et al., 2005, Neuropsychopharmacol 25:1132-6; Febo et al., 2004, J Neurosci Methods 139:167-76; Ferris et al., 2005, J Neurosci 25:149-56), nicotine (Skoubis et al., 2006, Neuroscience 137:583-91) and apomorphine (Chin et al., 2006, NeuroImage 33:1152-60; Zhang et al., 2000, Brain Res 852(2):290-6). Habituation to the scanning session is achieved by putting subjects through several simulated imaging studies.

When the rats were fully conscious, the restraining unit was placed into a black opaque tube mock scanner with a tape-recording of an MRI pulse sequence. This acclimation protocol lasted for 60 minutes in order to simulate the bore of the magnet and the imaging protocol. This procedure was repeated every other day for four days. With this procedure, rats show a significant decline in respiration, heart rate, motor movements, and plasma corticosteroid (CORT) when compared the first to the last acclimation periods (King et al., 2005, J. Neurosci. Methods 148(2):154-60).

Imaging Protocol

Experiments were conducted in a Bruker Biospec 4.7-T/40-cm horizontal magnet (Oxford Instrument, Oxford, U.K.) equipped with a Biospec Bruker console (Bruker, Billerica, Mass., U.S.A.) and a 20 G/cm magnetic field gradient insert (internal diameter=12 cm) capable of a 120 μs rise time (Bruker). Radiofrequency (RF) signals were sent and received with the dual coil electronics built into the animal restrainer (Ludwig et al., 2004, J. Neurosci. Methods 132(2):125-35). The volume coil for transmitting RF signal features an 8-element microstrip line configuration in conjunction with an outer copper shield. The arch-shaped geometry of the receiving surface coil provides excellent coverage and high signal-to-noise. To prevent mutual coil interference, the volume and surface coils were actively tuned and detuned.

Functional images were acquired using a multi-slice fast spin echo sequence. A single data acquisition included twelve (12), 1.2 mm slices collected in 6 seconds (field of view (FOV) 3.0 cm; data matrix 64×64; repetition time (TR) 1.43 sec, effective echo time (Eff TE) 53.3 msec, echo time (TE) 7 msec; rapid acquisition with relaxation enhancement (RARE) factor 16, number of excitations (NEX) 1). This sequence was repeated 100 times in a 10 minute imaging session, consisting of 5 minutes of baseline data followed by 5 minutes of stimulation data. At the beginning of each imaging session, a high resolution anatomical data set was collected using a RARE pulse sequence (12 slice; 1.2 mm; FOV 3.0 cm; 256×256; TR 2.1 sec; TE 12.4 msec; NEX 6; 7 minute acquisition time).

Motion Artifact

The experiments conducted in this work were a single epoch event-related design. To assess false activation due to subject motion, fMRI data were collected from awake rats (n=8) over a 10 minute scanning session in the absence of any stimulation. From these empirical data, a series of virtual fMRI data were numerically generated using a tri-linear interpolation algorithm with Gaussian noise and a preset amount of rigid body motion in random direction. The amount of motion introduced was in increment of 1/10 of a voxel (ca. 50 μm) up to 1 voxel (486 μm). The data was analyzed with statistical t-tests on each subject within their original coordinate system. On an average, approximately 3,500 voxels were tested for each subject. The control window was the first 50 time periods (5 minute), whereas the stimulation window was the remaining 50 time periods (5 minute) as described for the fMRI studies above. The t-test statistics used a 95% confidence level, two-tailed distributions, and heteroscedastic variance assumptions. In this case, a multiple comparison control (false detection rate) was not used to avoid suppression of any spurious activation. There was no significant change in BOLD signal or the number of activated voxels up to ca. 300 μm (or 6/10 of voxel) motion. Both number of voxels and percent BOLD signal increased dramatically as it approached one voxel of motion.

For each subject, rigid body motion in x-, y- and z-direction was computed with Stimulate software (Strupp, 1996, NeuroImage 3:S607) using center of intensity method. Standard deviation of this data measured how widely spread the motion was for each subject. A conservative criteria of 120 μm standard deviation of motion in any direction was set as acceptance criteria. In these experiments, motion in the z- and x-direction was small as compared to y-direction. Animals showing an average displacement exceeding 25% of the total in-plane (x-y) voxel resolution (>120 μm out of 468 μm) or more than 25% displacement in the slice (z) direction (>300 μm out of 1,200 μm slice thickness) were excluded. Most of the motion was in y-direction (64 μm±42 μm) and can be attributed to limitations in the design of the rat head holder.

Drug Administration

The subjects were first acclimated to the imaging protocol as described above. The rats were then pre-treated with the typical antipsychotic chlorpromazine (5 mg/kg) (GlaxoSmithKline, London, U.K.) or haloperidol (1 mg/kg) (Sandoz, Holzkirchen, Germany), the atypical antipsychotic clozapine (5 mg/kg) (Novartis, Basel, Switzerland) or olanzapine (5 mg/kg) (Eli Lilly, Indianapolis, Ind.) respectively), or cyclodextrin (Sigma-Aldrich, St. Louis, Mo.) in 0.9% saline solution as a control by intraperitoneal injection. The doses of anti-psychotics selected have been previously used in animal research and reflect doses used in clinical practice. To induce dopaminergic neurotransmission, the animals were challenged with intracerebroventricular injection of apomorphine (20 μg/10 μl) (Ipsen Ltd., Paris, France).

Data Analysis

Anatomy images for each subject were obtained at a resolution of 256²×12 slices and a FOV of 30 mm with a slice thickness of 1.2 mm. Subsequent functional imaging was performed at a resolution of 64²×12 slices with the same FOV and slice thickness. Each subject was registered to a segmented rat brain atlas. The alignment process was facilitated by an interactive graphic user interface. The affine registration involved translation, rotation, and scaling in all three dimensions, independently. The matrices that transformed the subject's anatomy to the atlas space were used to embed each slice within the atlas. All transformed pixel locations of the anatomy images were tagged with the segmented atlas major and minor regions creating a fully segmented representation of each subject. The inverse transformation matrix [Tᵢ]⁻¹ for each subject (i) was also calculated.

In this study, 12 brain slices were collected extending from the tip of the forebrain to the end of the cerebrum stopping at the midbrain just rostral to the cerebellum. Within these rostral/caudal boundaries, 83 minor volumes were delineated. In addition, brain areas were grouped into “major volumes” (e.g., amygdala, hippocampus, hypothalamus, cerebrum, etc.). The volume of activation (number of significant voxels) can be visualized in these 3D major and minor anatomical groupings.

Each scanning session consisted of 100 data acquisitions with a period of 6 seconds each for a total lapse time of 600 seconds or 10 minutes. The control window was the first 50 scan repetitions, while the stimulation window was scans 51-100 after the stimulation period. Statistical t-tests were performed on each subject within their original coordinate system. The baseline threshold was set at 2%. The t-test statistics used a 95% confidence level, two-tailed distributions, and heteroscedastic variance assumptions. As a result of the multiple t-test analyses performed, a false-positive detection controlling mechanism was introduced (Genovese et al., 2002, NeuroImage 15:870-8). This subsequent filter guaranteed that, on average, the false-positive detection rate was below the cutoff of 0.05. The formulation of the filter satisfied the following expression:

$${P}_{( i )} \leq \frac{i}{V} \frac{q}{c ( V )}$$

where P₍ᵢ₎ is the p value based on the t-test analysis. Each pixel (i) within the region of interest (ROI) containing (V) pixels was ranked based on its probability value. The false-positive filter value q was set to be 0.05 for the analyses, and the predetermined constant c(V) was set to unity, which is appropriate for data containing Gaussian noise such as fMRI data (Genovese et al., 2002, NeuroImage 15:870-8). These analysis settings provided conservative estimates for significance. Those pixels deemed statistically significant retained their percentage change values (stimulation mean minus control mean) relative to control mean. All other pixel values were set to zero.

A statistical composite was created for each group of subjects. The individual analyses were summed within groups. The composite statistics were built using the inverse transformation matrices. Each composite pixel location (i.e., row, column, and slice), premultiplied by [Tᵢ]⁻¹, mapped it within a voxel of subject (i). A tri-linear interpolation of the subject's voxel values (percentage change) determined the statistical contribution of subject (i) to the composite (row, column, and slice) location. The use of [Tᵢ]⁻¹ ensured that the full volume set of the composite was populated with subject contributions. The average value from all subjects within the group determined the composite value. The BOLD response maps of the composite were somewhat broader in their spatial coverage than in an individual subject. Thus, only average number of activated pixels that has the highest composite percent change values in particular ROI was displayed in composite map. Activated composite pixels are calculated as follows:

$$Activated Composite Pixels ROI ( j ) = \frac{{\sum }_{i = 1}^{N} Activated Pixels Subject ( i ) ROI ( j )}{N}$$

The composite percent change for the time history graphs for each region was based on the weighted average of each subject, as follows:

$$Composite Percent Change = \frac{{\sum }_{i = 1}^{N} Activated Pixel Subject ( i ) \times Percent Change ( i )}{Activated Composite Pixels }$$

where N is number of subjects.

B. Results

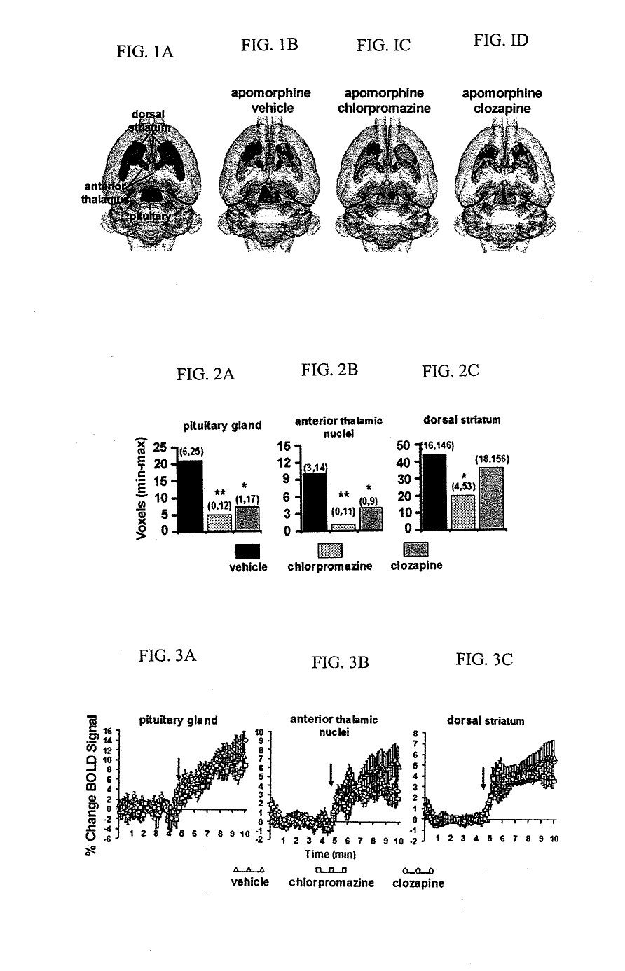

Three brain areas were identified that are differentially affected by both chlorpromazine (FIG. 1C) and clozapine (FIG. 1D). As depicted in FIGS. 1A-1D and FIGS. 2A-2C, apomorphine activated the pituitary gland (FIGS. 1A-1D; FIG. 2A), the anterior thalamic nuclei (FIGS. 1A-1C; FIG. 2B), and the dorsal striatum (FIGS. 1A-1D; FIG. 2C). The dorsal striatum, an area with a high density of dopamine receptors, remained active with clozapine but was reduced with chlorpromazine. Both chlorpromazine and clozapine caused a pronounced reduction in neuronal/glial activity in the pituitary gland and the anterior thalamic nuclei. These were the only two regions of the brain out of over 100 areas screened that showed this common profile.

To corroborate the ability of typical and atypical antipsychotics to reduce neuronal/glial activity in both the pituitary and anterior thalamus, the experiments were repeated using two different typical and atypical antipsychotics, haloperidol (1 mg/kg) (Sandoz, Holzkirchen, Germany) and olanzapine (5 mg/kg) (Eli Lilly, Indianapolis, Ind.) respectively). Apomorphine (20 μg/10 μl) was again administered to induce dopaminergic neurotransmission.

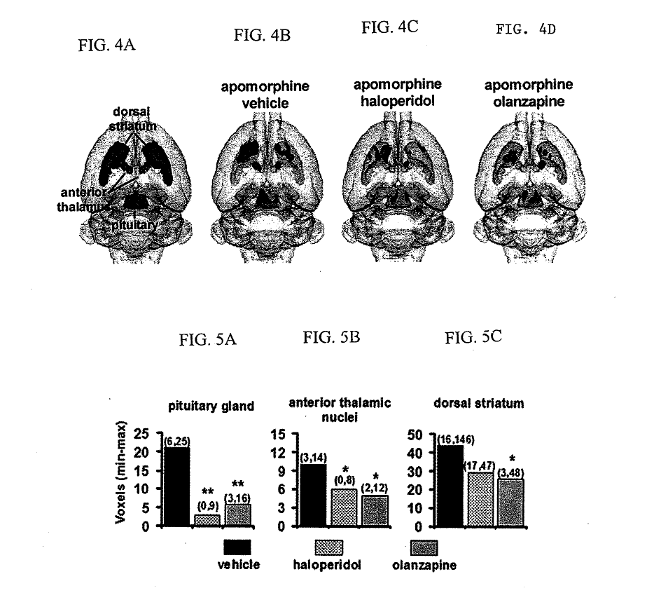

As shown in FIGS. 4A-4D and FIGS. 5A-5C, both haloperidol (FIG. 4C) and olanzapine (FIG. 4D) exhibited the common property of reducing apomorphine-induced neuronal/glial activity within the pituitary gland (FIG. 5A) and anterior thalamic nuclei (FIG. 5B) as chlorpromazine and clozapine. However, olanzapine, unlike clozapine, exhibited some dopamine blocking activity, a chemical characteristic confirmed by the reduction of neuronal/glial activity in the dorsal striatum (FIG. 5C). These results demonstrate that typical and atypical antipsychotics can reduce neuronal/glial activity in the anterior thalamic nuclei and pituitary gland after apomorphine-induced dopaminergic neurotransmission.

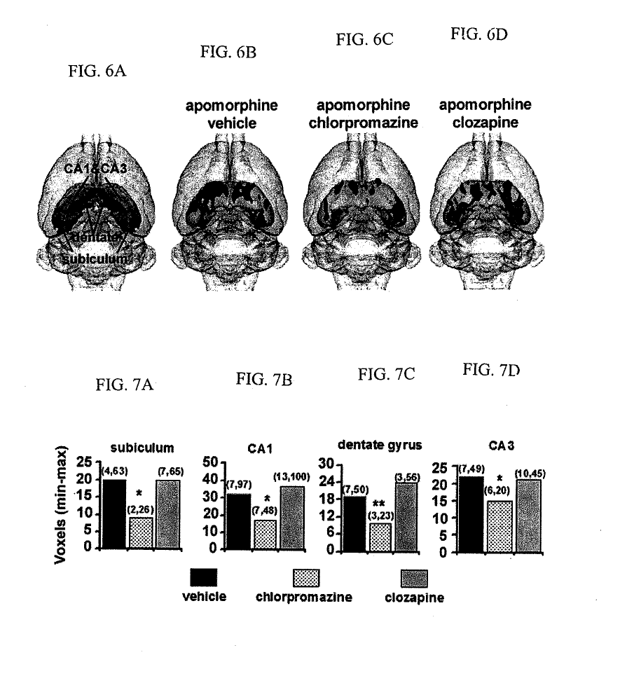

Over 100 rat brain regions were scanned using BOLD fMRI to identify brain regions with distinctive neuronal/glial activity of typical and atypical antipsychotics upon enhanced dopaminergic neurotransmission. In addition to the pituitary and the anterior thalamus, the hippocampus showed a unique neuronal/glial activity profile of antipsychotics upon apomorphine-induced dopamine activation (FIGS. 6A-6D; FIGS. 7A-7D). The hippocampus showed a selective reduction in activity to chlorpromazine (FIG. 6C), but not clozapine (FIG. 6D), throughout the hippocampus. In four distinct regions of the hippocampus (the subiculum (FIG. 7D), the CA1 region (FIG. 7B), the dentate gyms (FIG. 7C), and the CA3 region (FIG. 7D), there was a significant reduction in neuronal/glial activity upon dopaminergic activation when the rat was pre-treated with chlorpromazine. The hippocampus is thus a unique brain region that can be used to delineate between the two classes of antipsychotic drugs.

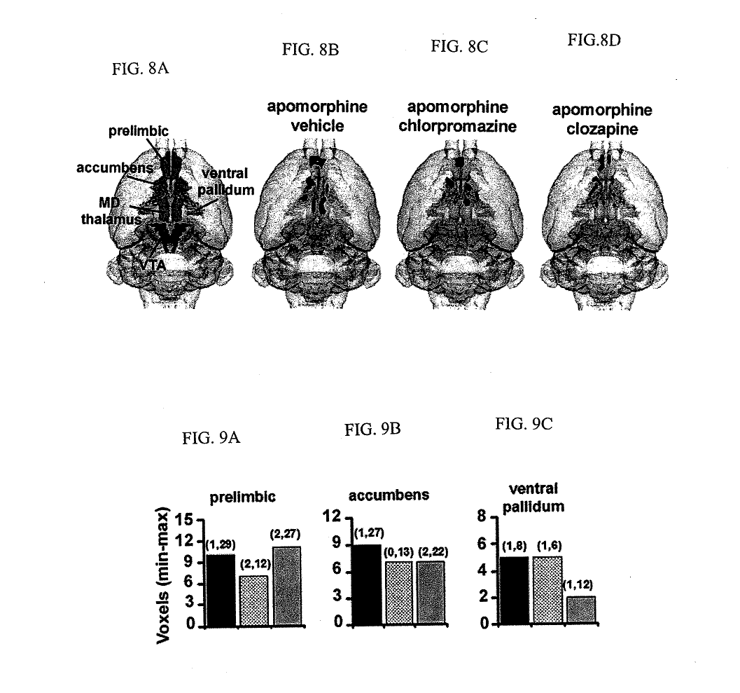

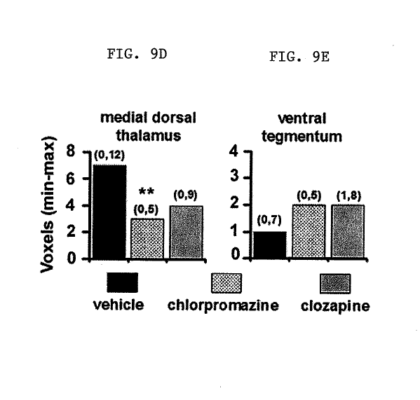

FIGS. 8A-8D and FIGS. 9A-9E demonstrated that other brain areas did not possess distinctive fingerprints of neuronal/glial activity upon dopaminergic neurotransmission after typical and atypical antipsychotics administration. These figures were representations of neuronal/glial activity within the mesocorticolimbic dopamine system (i.e., the reward pathway). This area was activated by apomorphine alone. However, neither typical nor atypical antipsychotics reduced the neuronal/glial activity in the mesocorticolimbic dopamine system, with the exception of the medial dorsal thalamus (FIG. 9D). These data thus provide another level of analysis showing regions of the brain that are not affected by the experimental manipulations.

In conclusion, specific and differential actions of typical and atypical actions in brain regions were observed specific to the spectral expression of the psychotic phenotype. In addition, the pituitary and anterior thalamus represent brain sites that are equally sensitive to the dopamine D2/D3 typical antipsychotics and the second generation 5-HT_{2A}/D2 relative-ratio atypical antipsychotics, in terms of their ability to decrease apomorphine-induced DA increases. In terms of abnormal DA function in psychosis, these brain regions represent a common “fingerprint” for general risk or pathology whereby treatment with either agent will produce decreases in symptoms associated with their function (i.e., stress and sensory filtering). In contrast, atypical and typical activity in the hippocampal formation demonstrated a neuroanatomical site in the brain where selective memory deficits characteristic of psychosis may be resistant to atypical treatment, warranting alterations in treatment regimens. This brain region is thus a viable candidate region for the investigation of antipsychotic indications for novel compounds.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for identifying a typical antipsychotic drug, comprising:
a. pre-treating a conscious subject with an effective amount of a test neuroleptic;
b. measuring neuronal/glial activity in the hippocampus of the subject by functional imaging;
c. administering a drug that activates dopaminergic neurotransmission to the subject sufficient to alter measurable subject behavior;
d. measuring neuronal/glial activity in the hippocampus of the subject after administration of the drug that activates dopaminergic neurotransmission; and
e. comparing the neuronal/glial activity in the hippocampus of the subject prior to and subsequent to the administration of the drug that activates dopaminergic neurotransmission,
a decrease in neuronal/glial activity in the hippocampus indicating that the test neuroleptic is a typical antipsychotic drug.

2. The method of claim 1, wherein the subject is a mammal.

3. The method of claim 1, wherein the drug that activates dopaminergic neurotransmission is a psychostimulant.

4. The method of claim 3, wherein the psychostimulant is selected from the group consisting of apomorphine, cocaine, amphetamine, methamphetamine, arecoline, methylphenidate, and mixtures thereof.

5. The method of claim 1, wherein functional magnetic resonance imaging (fMRI) measures the neuronal/glial activity.

6. A method for identifying an antipsychotic drug, comprising:
a. pre-treating a conscious subject with an effective amount of a test neuroleptic;
b. measuring neuronal/glial activity in the pituitary gland of the subject by functional imaging;
c. administering a drug that activates dopaminergic neurotransmission to the subject sufficient to alter measurable subject behavior;
d. measuring neuronal/glial activity in the pituitary gland of the subject after administration of the drug that activates dopaminergic neurotransmission; and
e. comparing the neuronal/glial activity in the pituitary gland of the subject prior to and subsequent to the administration of the drug that activates dopaminergic neurotransmission,
a decrease in neuronal/glial activity in the pituitary gland indicating that the test neuroleptic is an antipsychotic drug.

7. The method of claim 6, wherein the antipsychotic drug is a typical antipsychotic drug or an atypical antipsychotic drug.

8. The method of claim 6, wherein the subject is a mammal.

9. The method of claim 6, wherein the drug that activates dopaminergic neurotransmission is a psychostimulant.

10. The method of claim 9, wherein the psychostimulant is selected from the group consisting of apomorphine, cocaine, amphetamine, methamphetamine, arecoline, methylphenidate, and mixtures thereof.

11. The method of claim 6, wherein functional magnetic resonance imaging (fMRI) measures the neuronal/glial activity.

12. A method for identifying an antipsychotic drug, comprising:
a. pre-treating a conscious subject with an effective amount of a test neuroleptic;
b. measuring neuronal/glial activity in the anterior thalamic nuclei of the subject by functional imaging;
c. administering a drug that activates dopaminergic neurotransmission to the subject sufficient to alter measurable subject behavior;
d. measuring neuronal/glial activity in the anterior thalamic nuclei of the subject after administration of the drug that activates dopaminergic neurotransmission; and
e. comparing the neuronal/glial activity in the anterior thalamic nuclei of the subject prior to and subsequent to the administration of the drug that activates dopaminergic neurotransmission,
a decrease in neuronal/glial activity in the anterior thalamic nuclei indicating that the test neuroleptic is an antipsychotic drug.

13. The method of claim 12, wherein the antipsychotic drug is a typical antipsychotic drug or an atypical antipsychotic drug.

14. The method of claim 12, wherein the subject is a mammal.

15. The method of claim 12, wherein the drug that activates dopaminergic neurotransmission is a psychostimulant.

16. The method of claim 15, wherein the psychostimulant is selected from the group consisting of apomorphine, cocaine, amphetamine, methamphetamine, arecoline, methylphenidate, and mixtures thereof.

17. The method of claim 12, wherein functional magnetic resonance imaging (fMRI) measures the neuronal/glial activity.
